(19) United States
(12) Patent Application Publication
(10) Pub. No.: US 2017/0251981 A1
(43) Pub. Date: Sep. 7, 2017

(54) METHOD AND APPARATUS OF PROVIDING DEGREE OF MATCH BETWEEN BIOSIGNALS

(71) Applicant: Samsung Electronics Co., Ltd., Suwon-si (KR)

(72) Inventor: Sangyun PARK, Hwaseong-si (KR)

(73) Assignee: Samsung Electronics Co., Ltd., Suwon-si (KR)

(21) Appl. No.: 15/226,561

(22) Filed: Aug. 2, 2016

(30) Foreign Application Priority Data
Mar. 2, 2016 (KR) 10-2016-0025068

(51) Int. Cl. A61B 5/00; G06F 19/00; A61B 5/024; A61B 5/0488; A61B 5/0245

(52) U.S. Cl. CPC A61B 5/7275; A61B 5/0488; A61B 5/0245; A61B 5/02416; A61B 5/6802; G06F 19/345; G06F 19/3406; A61B 2562/0219

(57) ABSTRACT

A method and an apparatus of providing a degree of match between biosignals are provided. A method of providing a degree of match between biosignals involves receiving biosignals corresponding to users, calculating a degree of match between the biosignals, and providing the calculated degree of match between the biosignals to one or more users.

### CROSS-REFERENCE TO RELATED APPLICATIONS

This application claims the benefit under 35 USC 119(a) of Korean Patent Application No. 10-2016-0025068 filed on Mar. 2, 2016, at the Korean Intellectual Property Office, the entire disclosure of which is incorporated herein by reference for all purposes.

### BACKGROUND

1. Field

The following description relates to a method and an apparatus of providing a degree of match between biosignals of users and providing the degree of match to the users.

2. Description of Related Art

With the recent development of portable electronic devices, electronic apparatuses have been used to determine whether a user is making a motion. For example, an electronic apparatus may be used to determine an amount of exercise performed by a user in a health management and exercise management application. However, the apparatuses are not suitable for managing the amount of exercise performed by a plurality of users or to monitor motions of the plurality of users since the apparatuses are limited in that they are only designed to monitor an amount of exercise performed by an individual.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method of providing a degree of match between biosignals involves receiving biosignals corresponding to users, calculating a degree of match between the biosignals, and providing the calculated degree of match between the biosignals.

The calculating may be performed by a processor of a matching apparatus.

The calculating of the degree of match may be performed based on phase delays between the biosignals.

The calculating of the degree of match may involve detecting envelopes of waveforms corresponding to the biosignals, extracting feature points from the envelopes, calculating time differences between the feature points extracted from the envelopes, and calculating the degree of match between the biosignals based on the time differences between the feature points.

The feature points may include at least one of a maximum point, a minimum point, a peak point, a valley point, an inflection point, a slope maximum point, or a minimum slope point of a signal waveform corresponding to each of the envelopes.

The biosignals may include electromyographic (EMG) signals of the users, and the calculating of the degree of match may involve calculating an average value of EMG signals of at least a portion of the users.

The calculating of the degree of match may involve calculating a degree of match between an EMG signal of one of the users and the average value of the EMG signals, and calculating a synchronization degree of a motion pattern between the one of the users and at least a portion of the users based on the degree of match between the EMG signal of the one of the users and the average value of the EMG signals.

The providing of the calculated degree of match may involve providing a degree of match between a biosignal of one of the users and an average value of the biosignals in response to the degree of match between the biosignal of the one of the users and the average value of the biosignals being less than or equal to a preset reference.

The general aspect of the method may further involve quantifying a degree of a difference and a degree of match between a biosignal of one of the users and the biosignals of the users.

The calculating of the degree of match may involve indexing a degree of match among biosignals corresponding to each of muscles of the users corresponding to two or more body parts.

The general aspect of the method may further involve performing signal processing on the biosignals to remove a noise, and the calculating of the degree of match may involve calculating the degree of match based on the biosignals from which the noise is removed.

The general aspect of the method may further involve calculating an average value of the biosignals and a dispersion degree of the biosignals.

The biosignals may include heartbeat signals of the users, and the calculating of the average value of the biosignals and the dispersion degree of the biosignals may involve estimating an activity degree of the users based on an average value of the heartbeat signals and a dispersion degree of the heartbeat signals.

The estimating of the activity degree of the users may involve quantifying a degree of difference between an activity degree of one of the users and an average value of activity degrees of at least a portion of the users based on an average value and a dispersion degree of a heartbeat signal of the one of the users and heartbeat signals of at least a portion of the users.

The biosignals may be sensed using at least one of a strain sensor, an EMG sensor, an electrocardiogram (ECG) sensor, a photoplethysmography (PPG) sensor, a heartbeat sensor, an acceleration sensor, or a gyro sensor.

In another general aspect, a non-transitory computer-readable medium storing program instructions for controlling a processor to perform the aspects of the method described above is provided.

In yet another general aspect, an apparatus for providing a degree of match between biosignals includes a communication interface configured to receive biosignals corresponding to users, and a processor configured to calculate a degree of match between the biosignals and provide the calculated degree of match between the biosignals.

The processor may be configured to detect envelopes of waveforms corresponding to the biosignals, extract feature points from the envelopes, and calculate the degree of match of the biosignals based on time differences between the feature points.

The biosignals may include electromyographic (EMG) signals of the users, and the processor is configured to calculate an average value of EMG signals of at least a portion of the users and calculate a synchronization degree of a motion pattern between one of the users and at least a portion of the users based on the degree of match between an EMG signal of the one of the users and the average value of the EMG signals.

The processor may be configured to perform signal processing on the biosignals to remove a noise, and calculate the degree of match between the biosignals on which the signal processing is performed.

The general aspect of the apparatus may further include a sensor configured to sense the biosignals, wherein the sensor includes at least one of a strain sensor, an EMG sensor, an electrocardiogram (ECG) sensor, a photoplethysmography (PPG) sensor, a heartbeat sensor, an acceleration sensor, or a gyro sensor.

Other features and aspects will be apparent from the following detailed description, the drawings, and the claims.

### BRIEF DESCRIPTION OF THE DRAWINGS

FIG. 1A illustrates an example of an apparatus for providing a degree of match, and FIG. 1B illustrates examples of devices including the apparatus.

FIG. 2 is a flowchart illustrating an example of a method of providing a degree of match.

FIG. 3 is a flowchart illustrating an example of a method of calculating a degree of match of biosignals.

FIG. 4 is a flowchart illustrating another example of a method of providing a degree of match.

FIG. 5 is a flowchart illustrating still another example of a method of providing a degree of match.

FIG. 6 illustrates an example of a method of providing a calculated degree of match.

FIG. 7 illustrates further example of a method of providing a calculated degree of match.

FIG. 8 is a flowchart illustrating still another example of a method of providing a degree of match.

FIG. 9 is a block diagram illustrating an example of a system for providing a degree of match.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of this disclosure. For example, the sequences of operations described herein are merely examples, and are not limited to those set forth herein, but may be changed as will be apparent after an understanding of this disclosure, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of this disclosure.

Structural or functional descriptions of examples in the present disclosure are merely intended for the purpose of describing examples and the examples may be implemented in various forms and should not be construed as being limited to those described in the present disclosure.

Although terms of “first” or “second” are used to explain various components, the components are not limited to the terms. These terms are used only to distinguish one component from another component. For example, a “first” component may be referred to as a “second” component, or similarly, the “second” component may be referred to as the “first” component within the scope of the right according to the concept of the present disclosure.

It should be noted that if it is described in the specification that one component is “connected,” “coupled,” or “joined” to another component, a third component may be “connected,” “coupled,” and “joined” between the first and second components, although the first component may be directly connected, coupled or joined to the second component. In addition, it should be noted that if it is described in the specification that one component is “directly connected” or “directly joined” to another component, a third component may not be present therebetween. Likewise, expressions, for example, “between” and “immediately between” and “adjacent to” and “immediately adjacent to” may also be construed as described in the foregoing.

As used herein, the singular forms are intended to include the plural forms as well, unless the context clearly indicates otherwise. It will be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

In the following description, examples are set out which may be used to provide a degree of match of biosignals corresponding to users. Examples may be implemented in various products such as, personal computers (PC), laptop computers, tablet computers, smartphones, smart home appliances, wearable devices and the like. According to one example, a synchronization degree (a degree of match based on time) of motion patterns performed by users may be calculated based on biosignals of the users detected in smartphones, mobile devices, smart home systems, wearable devices and the like. In another example, feedbacks and information on an exercise effect may be provided to a plurality of users who are using an identical exercise program based on a calculated synchronization degree of motion patterns performed by the users while they are exercising. Hereinafter, various examples will be described in detail with reference to the accompanying drawings. In the drawings, like reference numerals refer to like elements.

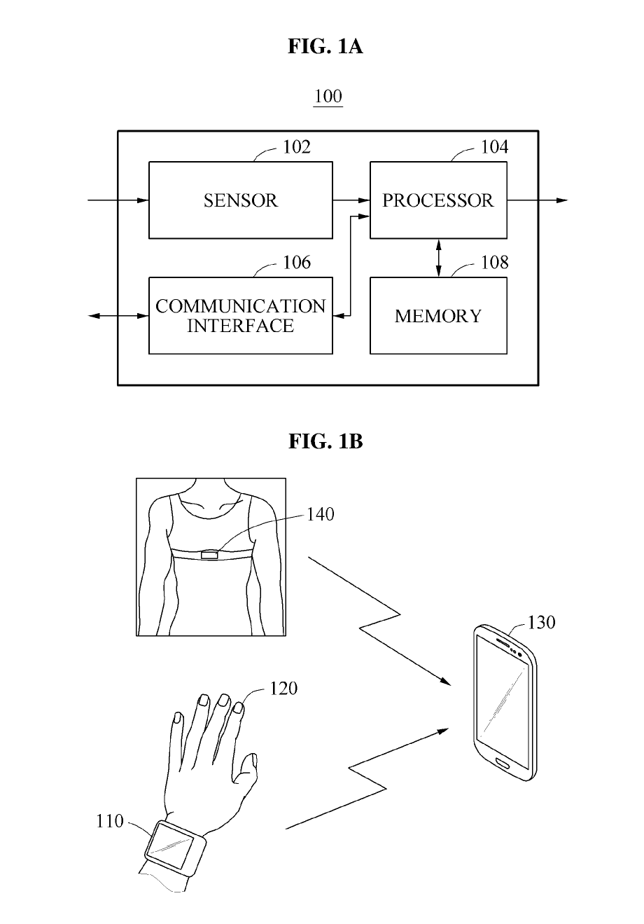

FIG. 1A illustrates an example of an apparatus for providing a degree of match, and FIG. 1B illustrates examples of devices including the apparatus. FIG. 1A is a block diagram illustrating an example of an apparatus 100, hereinafter also referred to as a matching apparatus 100, for providing a degree of match, and FIG. 1B illustrates devices including the matching apparatus 100.

Referring to FIG. 1A, the matching apparatus 100 includes a sensor 102, a processor 104, a communication interface 106, and a memory 108. In this example, the sensor 102, the processor 104, the communication interface 106, and the memory 108 communicate with each other via a bus (not shown). However, the method of communication between the sensor 102, the processor 104, the communication interface 106, and the memory 108 are not limited thereto.

The sensor 102 senses a biosignal of a user(s). The sensor 102 may include one sensor or a plurality of sensors. In this example, the sensor 102 includes at least one of a strain sensor, an EMG sensor, an electrocardiogram (ECG) sensor, a sensor configured to measure a photoplethysmography (PPG), a heartbeat sensor, an acceleration sensor, or a gyro sensor, a body temperature sensor, or a sensor configured to measure a change in a blood flow rate using an ultrasonic Doppler scheme and a laser Doppler scheme. Also, the sensor 102 includes a global positioning system (GPS) sensor and an inertial sensor, for example, a tilt sensor, a shock sensor, a gyro sensor, and an acceleration sensor, configured to sense a motion of a user. However, the scope of the examples is not limited thereto, and the sensor 102 may include various other types of sensors. A biosignal may refer to a signal of a living organism that may be continually measured or monitored, such as an electromyographic (EMG) signal, a heartbeat signal, skin conductance, and the like; however, the examples of biosignals are not limited thereto.

The processor 104 calculates a degree of match between a biosignal corresponding to a user sensed by the sensor 102 and biosignals corresponding to users received through the communication interface 106, and provides the calculated degree of match.

The processor 104 detects envelopes of waveforms corresponding to the biosignals, extracts feature points from the envelopes, and calculates the degree of match between the biosignals based on time differences between the feature points.

The processor 104 calculates an average value of EMG signals of at least a portion of the users. The processor 104 calculates a synchronization degree of a motion pattern between any one of the users and at least a portion of the users based on a degree of match between an EMG signal of any one of the users and the average value of the EMG signals.

The processor 104 performs signal processing on the biosignals for removing a noise and calculates a degree of match of the biosignals on which the signal processing is performed.

The communication interface 106 receives information from an external device, or provides the degree of match calculated by the processor 104 for the external device.

The memory 108 stores the biosignal of the user sensed by the sensor 102 and the degree of match of the biosignals calculated by the processor 104. The memory 108 includes a volatile memory and a non-volatile memory.

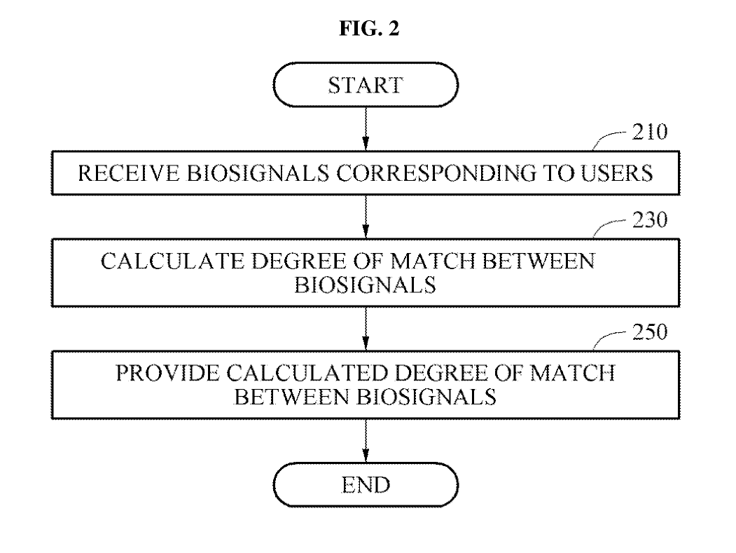

The processor 104 performs at least one method described with reference to FIGS. 2 through 9. The processor 104 executes a program and controls the matching apparatus 100. A program code executed by the processor 104 is stored in the memory 108. The matching apparatus 100 may be connected to an external device (for example, a PC or a network) via an input/output device (not shown), and may exchange data with the external device.

At least one method described with reference to FIGS. 2 through 9 may be implemented in a chip or an application operating in a processor included in a tablet computer, a smartphone or a wearable device and may be included in a smartphone or a wearable device.

FIG. 1B illustrates an example of a wearable device 110 that communicates with a mobile device 130, and a garment 140 of a user 120 that communicates with the mobile device 130. The matching apparatus 100 may be included in the wearable device 110, the mobile device 130, and/or the garment 140. The garment 140 of the user 120 may include an EMG sensor(s) implemented by way of a flexible conductive textile or a stretchable conductive textile, for example.

In an example, the matching apparatus 100 is mounted in the wearable device 110. In this example, the wearable device 110 may be a wrist wearable device having a shape of a watch or a bracelet, or may have a shape of a necklace, a chest belt, a patch or other shapes.

When the user 120 is wearing the wearable device 110 in daily life, the matching apparatus 100 calculates a degree of match between biosignals of multiple users based on the biosignals sensed through a plurality of wearable devices 110 or garments 140 of the users 120. The matching apparatus 100 receives a biosignal corresponding to a user by a sensor configured to sense a motion of the user included in the wearable device 110 or the garment 140 of a user 120.

The wearable device 110 or the garment 140 of a user 120 that includes a matching apparatus 100 interoperates with a mobile device 130, and shares data with the mobile device 130. For example, a degree of match of biosignals calculated based on a biosignal sensed by the user 120 through the wearable device 110 or the garment 140 of the user 120 and biosignals sensed from other users may be transmitted to the mobile device 130.

In another example, the processor 104 of the matching apparatus 100 is mounted in the mobile device 130, and the sensor 102 is mounted in the wearable device 110 or the garment 140 of the user 120. The wearable device 110 is worn on a body part, for example, a wrist or a chest, of the user 120, and measures a biosignal of the user 120 from the body part. The wearable device 110 amplifies and filters the measured biosignal. The wearable device 110 transmits the measured biosignal to the mobile device 130. The matching apparatus 100 included in the mobile device 130 calculates a degree of match of biosignals of users based on a heartbeat received from the wearable device 110 or the garment 140 of the user 120 and provides the degree of match.

In this example, the wearable device 110, the garment 140 of the user 120, and the mobile device 130 are connected to each other via a wireless link. The wearable device 110, the garment 140 of the user 120, and the mobile device 130 each include a wireless Internet interface and a local area communication interface. The wireless Internet interface may include, for example, a wireless local area network (WLAN) interface, a wireless fidelity (Wi-Fi) direct interface, a Digital Living Network Alliance (DLNA) interface, a wireless broadband (WiBro) interface, a World Interoperability for Microwave Access (WiMAX) interface, or a high speed downlink packet access (HSDPA) interface. The local area communication interface may include, for example, a Bluetooth interface, a radio frequency identification (RFID) interface, an infrared data association (IrDA) interface, a ultra-wideband (UWB) interface, a ZigBee interface, or a near field communication (NFC) interface.

The mobile device 130 may be implemented in the form of a tablet computer, a smartphone, a personal digital assistant (PDA), or the like. In yet another example, the mobile device 130 may be a network device such as a server. For example, the mobile device 130 may be a single server computer, a system similar to the server computer, at least one server bank, or a server “cloud” distributed between different geographical positions.

The mobile device 130 receives various biosignals in addition to an EMG signal and a heartbeat signal through the wearable device 110, the garment 140 of the user 120 or other measurement devices.

FIG. 2 is a flowchart illustrating an example of a method of providing a degree of match between biosignals. Referring to FIG. 2, in operation 210, an apparatus for providing a degree of match, hereinafter also referred to as a matching apparatus, receives biosignals of a plurality of users. For example, the biosignals may be sensed by various sensors included in the matching apparatus or sensed by a plurality of wearable devices or garments of users. The wearable devices and the garments of the users are differentiated from the matching apparatus. In this example, a garment of a user includes an electromyographic (EMG) sensor(s) the EMG sensor(s) or the like that may be implemented by way of a flexible conductive textile or a stretchable conductive textile. In this example, the biosignals may be EMG signals and heartbeat signals. For example, “the biosignals corresponding to the users” may be understood as biosignals measured or sensed from each user, and may include a first biosignal sensed from a first user and a second biosignal sensed from a second user.

In operation 230, the matching apparatus calculates a degree of match of the biosignals. The matching apparatus calculates the degree of match based on phase delays between the biosignals. The matching apparatus calculates an average value of EMG signals of at least a portion of the users. “At least a portion of users” may be understood as including a portion of users or all of the users.

In an example, the matching apparatus quantifies a degree of a difference between a biosignal of any one user and biosignals of all other users. The matching apparatus calculates a degree of match of biosignals corresponding to muscles, for example, brachial muscles, femoral muscles, and abdominal muscles, of a portion of body parts of the users by indexing or digitizing the degree of match as a value of each of the muscles of the portion of the body parts. That is, the degree of match or mismatch among the biosignals corresponding to muscles, for example, brachial muscles, femoral muscles, and the like, may be quantified into a value corresponding to each of the muscles.

In operation 250, the matching apparatus provides the calculated degree of match of the biosignals. The matching apparatus may provide the calculated degree of match for any one of the users or provide the degree of match for all of the users. The matching apparatus may provide the calculated degree of match through a wearable device of each user or provide the calculated degree of match through an additional display viewed by all users.

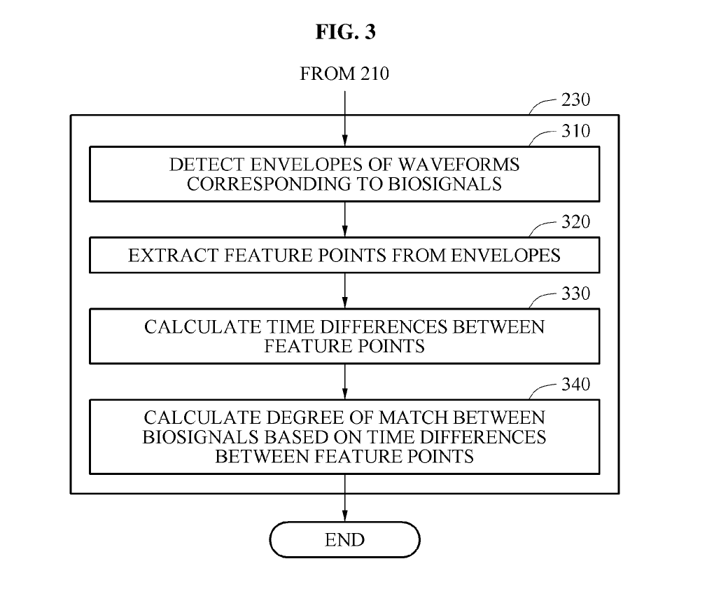

FIG. 3 is a flowchart illustrating an example of a method of calculating a degree of match of biosignals. Referring to FIG. 3, in operation 310, a matching apparatus detects envelopes of waveforms corresponding to biosignals. To detect the envelopes of waveforms from the biosignals, various methods may be used. For example, a method using a simple manual hardware such as an RC envelope detector, a method using an active hardware implemented by an integrator utilizing an operational amplifier, or a method using a low pass filter implemented as software may be used. It is desirable that the envelopes have a cut off frequency from 1 hertz (Hz) to 5 Hz in accordance with an example.

In operation 320, the matching apparatus extracts feature points from the envelopes. For example, the matching apparatus extracts the feature points by performing primary differentiation or secondary differentiation on a waveform of each of the envelopes.

The feature points include, for example, a maximum point, a minimum point, a peak point, a valley point, an inflection point, a slope maximum point, or a minimum slope point of a signal waveform of each of the envelopes. However, a type of a feature point is not limited thereto.

In operation 330, the matching apparatus calculates time differences between the feature points extracted in operation 320.

In operation 340, the matching apparatus calculates a degree of match of the biosignals based on the time differences between the feature points calculated in operation 330.

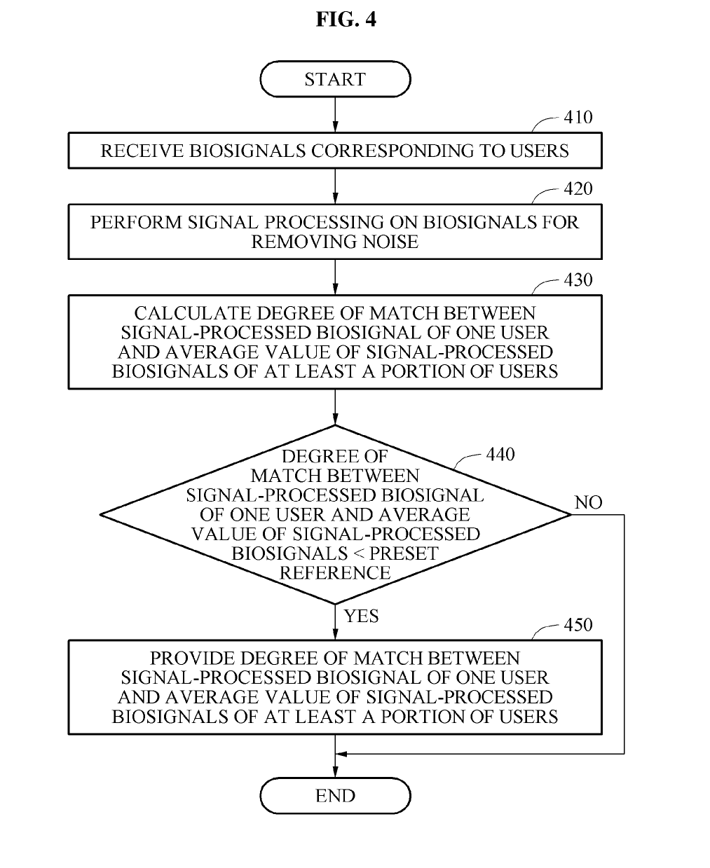

FIG. 4 is a flowchart illustrating another example of a method of providing a degree of match between biosignals. Referring to FIG. 4, in operation 410, a matching apparatus receives biosignals corresponding to users.

In operation 420, the matching apparatus performs signal processing on the biosignals for removing a noise. The biosignals collected through various paths may have various noise sources. The matching apparatus removes the noise using, for example, a low pass filter and a high pass filter.

In operation 430, the matching apparatus calculates a degree of match between a signal-processed biosignal of one user among the users and an average value of signal-processed biosignals of at least a portion of the users.

In operation 440, the matching apparatus determines whether the degree of match calculated in operation 430, that is, the degree of match between the signal-processed biosignal of the one user and the average value of the signal-processed biosignals, is less than a preset reference. Based on a determination that the degree of match calculated in operation 430 is greater than or equal to the preset reference, the matching apparatus terminates an operation.

Based on the determination that the degree of match calculated in operation 430 is less than the preset reference, the matching apparatus provides the degree of match between the signal-processed biosignal of the one use and the average value of the signal-processed biosignals of at least a portion of the users in operation 450.

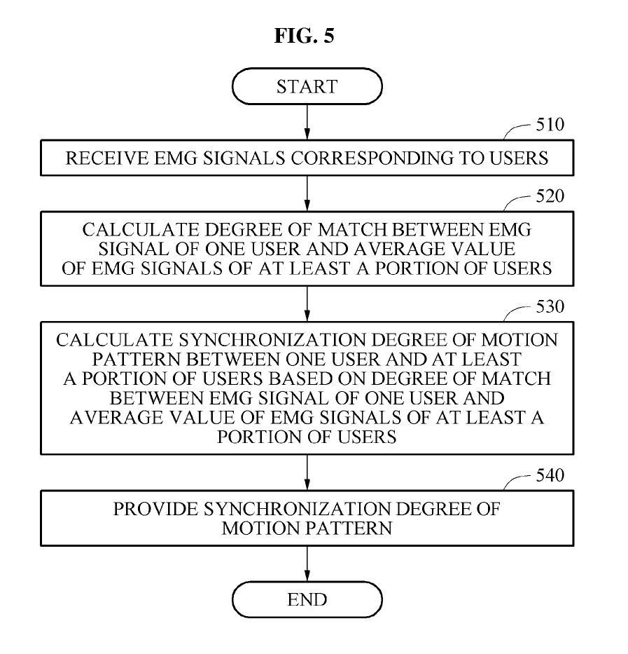

FIG. 5 is a flowchart illustrating still another example of a method of providing a degree of match. Referring to FIG. 5, in operation 510, a matching apparatus receives electromyographic (EMG) signals corresponding to users. For example, the plurality of users may be moving or exercising in response to the rhythm of an identical piece of music or identical exercise program.

In operation 520, the matching apparatus calculates a degree of match between an EMG signal of one of the users and an average value of EMG signals of at least a portion of the users.

In operation 530, the matching apparatus calculates a synchronization degree of a motion pattern between the one user and at least a portion of the users based on the degree of match between the EMG signal of the one user and the average value of the EMG signals of at least a portion of the users.

In operation 540, the matching apparatus provides the synchronization degree of the motion pattern calculated in operation 530.

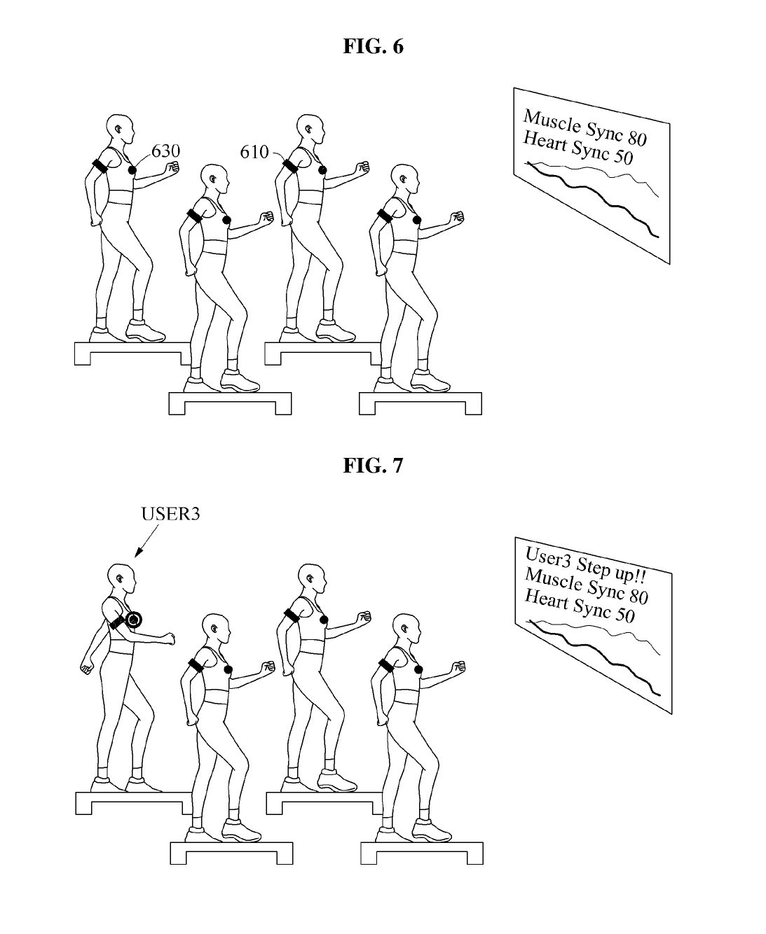

FIG. 6 illustrates an example of a method of providing a calculated degree of match. Referring to FIG. 6, users perform a group exercise.

Each of the users participating in the group exercise may wear a wearable device, for example, a wearable device 610, including various sensors, or a garment, for example, a garment 630, including various sensors implemented by way of a flexible conductive textile or a stretchable conductive textile. Biosignals such as electromyographic (EMG) signals and heartbeat signals according to a movement of each muscle of a portion of body parts of a user(s) may be sensed by the wearable device 610 or the garment 630.

The matching apparatus may measure a degree of match or a synchronization degree of biosignals of the users by receiving a biosignal of each of the users. The matching apparatus may index or digitize the measured degree of match to provide feedback for users or an instructor, thereby maximizing a feedback effect and an exercise effect for an exercise program.

The matching apparatus may index or digitize the degree of match of the EMG signals or the heartbeat signals of all users participating in the group exercise as a single value, for example, “muscle sync 80” or “heart sync 50”. Alternatively, the matching apparatus may index the degree of match or the synchronization degree of EMG signals of all members of a group as a value for each of muscles, for example, brachial muscles, femoral muscles, and abdominal muscles.

FIG. 7 illustrates a further example of a method of providing a calculated degree of match. Referring to FIG. 7, users perform a group exercise.

A matching apparatus indicates a synchronization degree between a motion of an individual participating in the group exercise and motions of all users in a group. The matching apparatus estimates an activity degree or a degree of match between a biosignal of any one user and the biosignals of all of the users of the group to which the one user belongs. For example, the biosignal may be obtained by an electromyographic (EMG) sensor, an acceleration sensor, or a strain sensor. The matching apparatus may identify and encourage a user of which a degree of match or an activity degree is less than a preset reference.

The matching apparatus may evaluate the activity degree of all of the users in the group by calculating an average value and a dispersion degree of heartbeat signals. The matching apparatus may use an activity tracker based on an accelerometer. The matching apparatus may calculate the average value (or an average of activity degrees) and the dispersion degree of the heartbeat signals to provide feedback. Thus, the matching apparatus evaluates the activity degree of all of users in the group or indexes (digitizes/quantifies) the degree of match of the activity degree of each user in the group. The matching apparatus may also provide feedback by indexing (digitizing/quantifying) a degree of a difference between the activity degree of an individual and the activity degree of all of the users in the group.

The matching apparatus identifies and encourages the user of which the activity degree is relatively low, or identifies a user of which the activity degree is relatively high to cool the user down.

For example, in response to a user 3 moving slowly compared to other users in the group, the synchronization degree or the activity degree of the user 3 may be less than the preset reference in comparison with an average value of all users in the group, for example, 85% of an entire average value. The matching apparatus may display the synchronization degree or the activity degree of the user 3 on a display, or provide an encouragement phrase, for example, “user 3 step up!,” either visually on a display or audibly by a sound, in order to encourage the user 3 to increase the activity level.

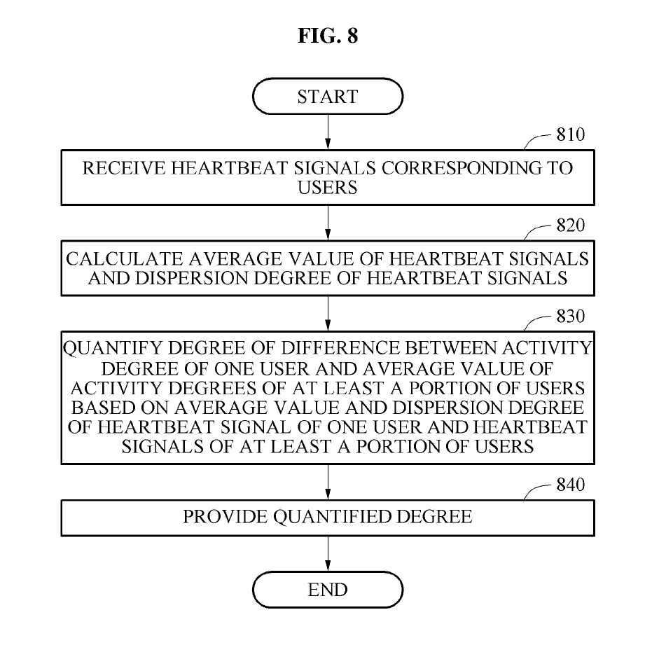

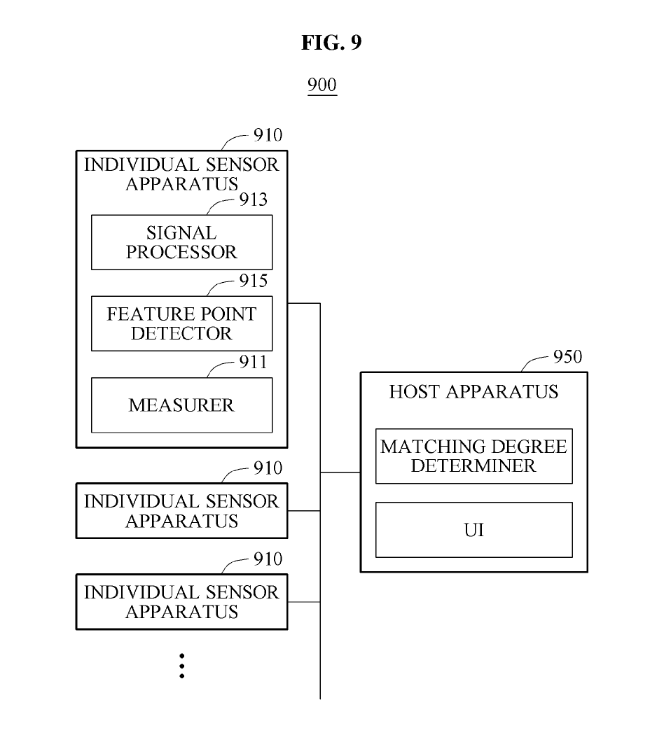

FIG. 8 is a flowchart illustrating yet another example of a method of providing a degree of match between biosignals. Referring to FIG. 8, in operation 810, a matching apparatus receives heartbeat signals corresponding to users. The heartbeat signals may be sensed by wearable devices or garments that include a heartbeat sensor implemented with a flexible conductive textile or a stretchable conductive textile.

In operation 820, the matching apparatus calculates an average value of the heartbeat signals and a dispersion degree of the heartbeat signals. The matching apparatus calculates an average value and a dispersion degree of heartbeat signals of at least a portion of the users. “The average value and the dispersion degree of the heartbeat signals of the at least a portion of the users” may be understood as including an average value of heartbeat signals of all users and a dispersion degree of the heartbeats of all users, an average value of heartbeat signals of a portion of users, and a dispersion degree of heartbeat signals of a portion of users. The matching apparatus performs signal processing (or preprocessing) on the heartbeat signals received in operation 810 for removing a noise, and calculates the average value and the dispersion degree based on a peak point and a valley point of the heartbeat signals on which signal processing is performed.

In operation 830, the matching apparatus quantifies a degree of a difference between an activity degree of any one of the users and an average value of activity degrees of at least a portion of the users based on the average value and the dispersion degree of a heartbeat signal of any one of the users and the heartbeat signals of at least a portion of the users calculated in operation 820. In operation 840, the matching apparatus provides the degree quantified in operation 830.

FIG. 9 is a block diagram illustrating an example of a system for providing a degree of match between biosignals. Referring to FIG. 9, a system 900 includes a plurality of individual sensor apparatuses 910 and a host apparatus 950.

The individual sensor apparatuses 910 collect and process a biosignal of each user. The host apparatus 950 combines the individual sensor apparatuses 910 and determines a degree of match or an activity degree. The host apparatus 950 may be a different kind of apparatus than the individual sensor apparatuses 910, but any one of the individual sensor apparatuses 910 may perform a role of the host apparatus 950.

Each of the individual sensor apparatuses 910 includes a measurer 911, a signal processor 913, and a feature point detector 915.

The measurer 911 measures or senses a biosignal such as an electromyographic

(EMG) signal and a heartbeat of a user. For example, the biosignal may be an EMG or heartbeat measured with an EMG sensor, a heartbeat sensor, an accelerometer, or a strain sensor. The measurer 911 may be implemented with, for example, a garment sensor that utilizes a conductive textile.

The signal processor 913 performs signal processing for removing a noise component from a signal measured by the measurer 911 through, for example, low pass filtering and high pass filtering.

The feature point detector 915 detects feature points from the signal from which the noise component is removed. The feature points include, for example, a maximum point, a minimum point, a peak point, a valley point, an inflection point, a slope maximum point, or a minimum slope point of the signal from which the noise component is removed. For example, an envelope of a signal waveform may be used to extract the feature points.

The host apparatus 950 calculates the degree of match by determining a degree to which the feature points received from each of the individual sensor apparatuses 910 temporally match or an amount of delay for each of the feature points. A delay of the feature points refers to a time difference between two corresponding feature points or a difference between time differences of each of feature points and an average value of the time differences.

The host apparatus 900 determines the degree of match of each of the individual sensor apparatuses 910 without the additional host apparatus 950, and feeds a determination result back through an additional user interface (UI). Each of the individual sensor apparatuses 910 may be in a wearable device or a garment sensor apparatus worn by each user.

As a non-exhaustive example only, a terminal/device/unit as described herein may be a mobile device, such as a cellular phone, a smart phone, a wearable smart device (such as a ring, a watch, a pair of glasses, a bracelet, an ankle bracelet, a belt, a necklace, an earring, a headband, a helmet, or a device embedded in clothing), a portable personal computer (PC) (such as a laptop, a notebook, a subnotebook, a netbook, or an ultra-mobile PC (UMPC), a tablet PC (tablet), a phablet, a personal digital assistant (PDA), a digital camera, a portable game console, an MP3 player, a portable/personal multimedia player (PMP), a handheld e-book, a global positioning system (GPS) navigation device, or a sensor, or a stationary device, such as a desktop PC, a high-definition television (HDTV), a DVD player, a Blu-ray player, a set-top box, or a home appliance, or any other mobile or stationary device configured to perform wireless or network communication. In one example, a wearable device is a device that is designed to be mountable directly on the body of the user, such as a pair of glasses or a bracelet. In another example, a wearable device is any device that is mounted on the body of the user using an attaching device, such as a smart phone or a tablet attached to the arm of a user using an armband, or hung around the neck of the user using a lanyard.

The matching apparatus, sensor, processor, communication interface, memory, sensor apparatus, signal processor, feature point detector, measurer, host apparatus, matching degree determiner, and user interface shown in FIGS. 1A, 1B and 9 that perform the operations described in this application are implemented by hardware components configured to perform the operations described in this application that are performed by the hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 2-8 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions in the specification, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media.

Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of this disclosure that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of providing a degree of match between biosignals, the method comprising:
receiving biosignals corresponding to users;
calculating a degree of match between the biosignals; and
providing the calculated degree of match between the biosignals.

2. The method of claim 1, wherein the calculating is performed by a processor of a matching apparatus.

3. The method of claim 1, wherein the calculating of the degree of match is performed based on phase delays between the biosignals.

4. The method of claim 1, wherein the calculating of the degree of match comprises:
detecting envelopes of waveforms corresponding to the biosignals;
extracting feature points from the envelopes;
calculating time differences between the feature points extracted from the envelopes; and
calculating the degree of match between the biosignals based on the time differences between the feature points.

5. The method of claim 4, wherein the feature points comprise at least one of a maximum point, a minimum point, a peak point, a valley point, an inflection point, a slope maximum point, or a minimum slope point of a signal waveform corresponding to each of the envelopes.

6. The method of claim 1, wherein the biosignals comprise electromyographic (EMG) signals of the users, and the calculating of the degree of match comprises calculating an average value of EMG signals of at least a portion of the users.

7. The method of claim 6, wherein the calculating of the degree of match comprises:
calculating a degree of match between an EMG signal of one of the users and the average value of the EMG signals; and
calculating a synchronization degree of a motion pattern between the one of the users and at least a portion of the users based on the degree of match between the EMG signal of the one of the users and the average value of the EMG signals.

8. The method of claim 1, wherein the providing of the calculated degree of match comprises providing a degree of match between a biosignal of one of the users and an average value of the biosignals in response to the degree of match between the biosignal of the one of the users and the average value of the biosignals being less than or equal to a preset reference.

9. The method of claim 1, further comprising:
quantifying a degree of difference and a degree of match between a biosignal of one of the users and the biosignals of the users.

10. The method of claim 1, wherein the calculating of the degree of match comprises indexing a degree of match among biosignals corresponding to each of muscles of the users corresponding to two or more body parts.

11. The method of claim 1, further comprising:
performing signal processing on the biosignals to remove a noise,
wherein the calculating of the degree of match comprises calculating the degree of match based on the biosignals from which the noise is removed.

12. The method of claim 1, further comprising:
calculating an average value of the biosignals and a dispersion degree of the biosignals.

13. The method of claim 12, wherein the biosignals comprise heartbeat signals of the users, and the calculating of the average value of the biosignals and the dispersion degree of the biosignals comprises estimating an activity degree of the users based on an average value of the heartbeat signals and a dispersion degree of the heartbeat signals.

14. The method of claim 13, wherein the estimating of the activity degree of the users comprises quantifying a degree of a difference between an activity degree of one of the users and an average value of activity degrees of at least a portion of the users based on an average value and between a dispersion degree of a heartbeat signal of the one of the users and heartbeat signals of at least a portion of the users.

15. The method of claim 1, wherein the biosignals are sensed using at least one of a strain sensor, an EMG sensor, an electrocardiogram (ECG) sensor, a photoplethysmography (PPG) sensor, a heartbeat sensor, an acceleration sensor, or a gyro sensor.

16. A non-transitory computer-readable medium storing program instructions for controlling a processor to perform the method of claim 1.

17. An apparatus for providing a degree of match between biosignals, the apparatus comprising:
a communication interface configured to receive biosignals corresponding to users; and
a processor configured to calculate a degree of match between the biosignals and provide the calculated degree of match between the biosignals.

18. The apparatus of claim 17, wherein the processor is configured to detect envelopes of waveforms corresponding to the biosignals, extract feature points from the envelopes, and calculate the degree of match of the biosignals based on time differences between the feature points.

19. The apparatus of claim 17, wherein the biosignals comprise electromyographic (EMG) signals of the users, and the processor is configured to calculate an average value of EMG signals of at least a portion of the users and calculate a synchronization degree of a motion pattern between one of the users and at least a portion of the users based on the degree of match between an EMG signal of the one of the users and the average value of the EMG signals.

20. The apparatus of claim 17, wherein the processor is configured to perform signal processing on the biosignals to remove a noise, and calculate the degree of match between the biosignals on which the signal processing is performed.

21. The apparatus of claim 17, further comprising:
a sensor configured to sense the biosignals,
wherein the sensor comprises at least one of a strain sensor, an EMG sensor, an electrocardiogram (ECG) sensor, a photoplethysmography (PPG) sensor, a heartbeat sensor, an acceleration sensor, or a gyro sensor.
